(19) United States
(12) Patent Application Publication
(10) Pub. No.: US 2025/0204971 A1
(43) Pub. Date: Jun. 26, 2025

(54) ELECTROSURGICAL LARYNGEAL WAND

(71) Applicants: Smith & Nephew, Inc., Memphis, TN (US); Smith & Nephew Orthopaedics AG, Zug (CH)

(72) Inventors: Patrick E. Barton, Buda, TX (US); Angela S. Crall, Austin, TX (US); Jeffrey S. Wrana, Austin, TX (US); Douglas G. Evans, Austin, TX (US)

(21) Appl. No.: 18/849,931

(22) PCT Filed: May 23, 2023
(86) PCT No.: PCT/US2023/023200

§ 371 Date: Sep. 23, 2024

Related U.S. Application Data
(60) Provisional application No. 63/345,064, filed on May 24, 2022.
(60) Provisional application No. 63/344,798, filed on May 23, 2022.

(51) Int. Cl. A61B 18/04; A61B 18/00

(52) U.S. Cl. CPC A61B 18/04; A61B 2018/00083; A61B 2018/00327; A61B 2218/007

(57) ABSTRACT

A bipolar electrosurgical wand for treating tissue along a patient airway. The wand includes a tubular end effector with an electrically insulative spacer, a return electrode, and an active electrode at its distal end. The active electrode includes an annular portion and a tip projection extending distally therefrom. The annular portion may be coextensive with the insulative spacer, and the tip projection may extend distally beyond a distal-most surface of the insulative spacer. Both the tip projection and annular portion may share a continuous top planar surface. The annular portion includes an aspiration opening therethrough for removing tissue debris from the target site.

### CROSS-REFERENCE TO RELATED APPLICATIONS

This application claims benefit to U.S. Provisional Patent Application 63/345,064, titled “Electrosurgical Laryngeal Wand”, filed May 24, 2022, commonly owned and herein incorporated by reference in its entirety. This application also claims benefit to U.S. Provisional Patent Application 63/344,798, titled “Electrosurgical Laryngeal Wand”, filed May 23, 2022, commonly owned and herein incorporated by reference in its entirety.

### FIELD

This application relates generally to methods and apparatus for accessing and treating tissue. More specifically disclosed, is an apparatus and associated method for electrosurgically treating a range of pathological conditions that affect the laryngeal and/or airway anatomy.

### BACKGROUND

Access and treatment of areas along a patient airway, around the larynx presents a unique set of challenges. For example, the airway is narrow, limiting device size. The airway is relative long, requiring a substantial length of device. Visibility at the end of the device may also be limited. Some of the tissues along the airway are sensitive to energy-based treatments, such that inadvertent treatment or simply contact with a hot surface may cause significant complications. For example, a polyp may require removal from a vocal cord, the vocal cord being particularly sensitive to heat. The tissues or pathologies along the airway are generally small and therefore overtreatment, including applying excessive energy may generally be a risk. Some procedures require a combination of both fine dissection and some larger scale debulking, often requiring multiple devices for a single procedure. Devices may be provided with suction to remove fluids and treated tissue from the treatment site and improve overall visibility, the suction pathways prone to clogging due to the overall size limitations of the devices.

Therefore, there is a need for a single device that addressed the shortcomings described above. There is a need for a single device that offers targeted tissue removal in narrow anatomies via electrosurgical treatment of tissue. There is a need for a single device that provides access to narrow anatomies while providing increased surgical field visualization. There is a need for a single device that limits inadvertent tissue damage. There is a need for a device that accesses a plurality of pathologies along the airway, that may also offer a plurality of tissue treatment modes, such as fine dissection and debulking.

### SUMMARY

An improved electrosurgical wand for treatment of a variety of pathologies along the patient airway, and more specifically tissues in and around the larynx. The improved wand may include a multifunctional treatment electrode that may both finely dissect tissues and/or debulk tissue. The wand, in cooperation with an electrosurgical controller may treat tissues by means of ablation as defined herein. A more detailed description of the ablation can be found in commonly assigned U.S. Pat. No. 5,697,882, the complete disclosure of which is incorporated herein by reference.

This electrode may include a planar treatment surface configured to debulk tissue along the larynx, via ablation. This electrode may also include an edge surface and/or distally projecting tip configured to dissect the tissue along the larynx via ablation. The wand may deliver an electrically conductive fluid to the target site and aspirate tissue, fluid and plasma by-products therefrom. The wand may be a handheld wand and thereby used directly by a clinician or may be configured to be controlled via a robotically controlled surgical setup. The wand may include improvements to suction openings and suction pathways to significantly reduce the potential for clogging the wand, as is prone to occur in related art wands.

A first example bipolar electrosurgical wand embodiment is disclosed herein, that includes a tubular end effector with an electrically insulative spacer, a return electrode, and an active electrode at a distal end thereof. The insulative spacer both supports and electrically insulates the active electrode from the return electrode. The active electrode includes an annular portion and a tip projection extending distally from the annular portion. The annular portion is coextensive with the insulative spacer and the tip projection extends distally beyond a distal-most surface of the insulative spacer. Both the tip projection and annular portion share a continuous top planar (flat) surface. The annular portion includes a 360-degree bounded hole therethrough that that is an aspiration opening for removing at least one of tissue, debris and fluid therethrough.

In some example embodiments, the tip projection may have a maximum lateral width that is less than half a corresponding maximum lateral width of the annular portion. The active electrode may define an outermost peripheral edge surface, that includes bilateral concave edge surfaces that are coextensive with each other at a transition from the annular portion to the tip projection and the bilateral concave edge surfaces may be coextensive with a distal-most end surface of the insulative spacer.

In some example embodiments, the aspiration opening may extend from the active electrode planar top surface through to a bottom surface of the active electrode at an incline angle to the planar top surface. This incline angle may deflect aspirated tissue debris proximally into an aspiration conduit that extends along the tubular end effector. This incline angle may be oriented such that an edge boundary of the aspiration opening coincident with the bottom surface is axially offset from the corresponding edge boundary of the aspiration opening coincident with the top surface. The bottom surface edge boundary may be proximally offset from the corresponding edge boundary of the aspiration opening at the top planar surface. This edge boundary coincident with the bottom surface provide an edge surface that further digests aspirated tissue that flows through the aspiration opening. This edge boundary at the bottom surface may also include at least on notch expending along the aspiration opening, the notch providing supplemental edge surfaces to further digest aspirated tissue that flows through the aspiration opening.

In some example embodiments, the aspiration opening cross section may include a proximal-most apex with a first radius of curvature and a distal most curved end that has a radius of curvature that is at least twice the first radius of curvature. These differences in radii may provide a large enough opening to aspirate tissue, but with sufficient localized restriction to manage tissue debris and clogging. The first radius of curvature may preferably diminish a plasma-remote zone that extends through the aspiration opening and the second radius of curvature may preferably provide an extended surface area for further digesting tissue that flows through the aspiration opening.

In some example embodiments, the return electrode may include bilateral arms that may extend around the distal-most end surface of the insulative spacer, defining distal facing surfaces of the return electrode coextensive with the active electrode tip projection. These bilateral arms may aid in plasma initiation at the tip projection.

Another bipolar electrosurgical wand embodiment disclosed herein may include a tubular end effector with a handle at a proximal end thereof, and a return electrode, an insulative spacer and an active electrode at a distal end thereof. The insulative spacer may support and electrically insulate the active electrode. The active electrode may include an annular portion with a tip projection extending distally from the annular portion; the annular portion, and the tip projection both sharing a continuous top planar surface. The annular portion may define an aspiration opening, forming a 360-degree bounded hole that extends from the top planar surface to a bottom surface of the active electrode. This aspiration opening defines a central axis that extends at an incline angle to the planar top surface. This aspiration opening incline angle defines surfaces and edge surfaces that both aids in further digesting any tissue debris that flows through the aspiration opening and also deflects the tissue debris towards an aspiration conduit that extends proximally along the tubular end effector.

In some example embodiments, the incline angle extends in a proximal direction from the planar top surface of the active electrode. The 360-degree bounded hole may define a curved wedge cross section, with a proximal-most apex having a first radius of curvature and a distal-most curved end that has a radius of curvature that is at least double the first radius of curvature.

In some example embodiments, the tip projection may be a maximum transverse width that is smaller than half of a maximum transverse width of the annular portion. The tip projection may define a free end projection, that extends beyond the insulative spacer.

An example method of electrosurgically treating a tissue along a patient airway is also disclosed. This method includes positioning an electrosurgical wand in a first orientation so that a planar top surface of an active electrode engages a first target tissue along the patient airway, the active electrode having an aspiration opening extending from the planar top surface to a bottom surface of the active electrode. The aspiration opening may define a central axis oriented at a non-perpendicular angle to the planar top surface so that a peripheral edge boundary of the aspiration opening coincident with the bottom surface is axially offset from a corresponding peripheral edge boundary at the planar top surface. While the wand is in the first orientation, electrical energy may be delivered to the active electrode and a return electrode of the electrosurgical wand, sufficient to form, responsive to this energy, a localized plasma proximate to the active electrode planar surface. This may debulk the first target tissue, by the localized plasma, to molecularly dissociate a portion of the first target tissue forming tissue debris. This tissue debris may be aspirated through the aspiration opening, and as the tissue debris flows through the aspiration opening, it may be further molecularly dissociated via the localized plasma formed at the peripheral edge boundary coincident with the bottom surface, responsive to the energy being delivered.

In some example methods, the wand may be moved to a second orientation, such that a projecting tip of the active electrode is directly adjacent a second target tissue of the tissue along the patient airway, the projecting tip defining a distal-most projection of the active electrode, extending parallel to and continuous with the planar top surface. While the electrosurgical wand is in this second orientation, electrical energy may be applied between the active electrode and the return electrode to form, responsive to the energy, a localized plasma proximate to the projecting tip. The second target tissue may be finely dissected, by ablating, with the localized plasma. While applying the electrical energy between the active electrode and the return electrode to form, responsive to the energy, a localized plasma proximate to the projecting tip, an electrically conductive fluid may flow out of a fluid delivery aperture proximally spaced from the active electrode along the wand external surface distal end and around to a distal facing portion of the return electrode coextensive with the projecting tip. The distal facing portion and the projecting tip are in close proximity to reduce an electrical bridge burden on the electrically conductive fluid and thereby reduce a time to initiate the localized plasma proximate the projecting tip.

In some example methods, while applying electrical energy between the active electrode and return electrode, adjacent tissues may be protected from inadvertent thermal effects adjacent a back-side of the wand distal end, the back side formed of a thermal heat shrink ceramic.

In some example methods the tissue debris flowing through the aspiration opening is deflected proximally and towards an aspiration conduit disposed along the electrosurgical wand, the deflecting with a distal inclined surface of the aspiration opening. This distal surface may extend parallel to the central axis.

Another example bipolar electrosurgical wand embodiment is disclosed herein, the wand including a tubular end effector carrying a bipolar electrode arrangement at a distal portion thereof. The bipolar electrode arrangement may include a first active electrode, a second active electrode and a return electrode. The first active electrode may have a first active electrode distal most treatment surface and the second active electrode may have a second active electrode distal most surface, and the first active electrode may slide axially between a first configuration and a second configuration, relative to the second active electrode. In the first configuration, the first and second active electrode distal-most treatment surfaces may be axially adjacent each other forming a single continuous tissue treatment surface that may electrosurgically treat tissue in a first mode. In the second configuration the first active electrode maybe axially distally offset from the second active electrode, forming a discontinuous tissue treatment surface with the second active electrode. The first active electrode alone may electrosurgically treat tissue in a second mode, different than the first mode while in the second configuration.

In some example embodiments, the first active electrode distal-most surface may be smaller than the second active electrode distal most surface. The second active electrode distal-most surface may define a surface area that is at least twice that of a corresponding surface area of the first active electrode distal-most surface. The first mode may be a debulking mode and the second mode may be a fine dissection mode. The first mode may be a coagulating mode, and the second mode may be a cutting mode. In the second mode, the second active electrode may be dormant or electrically inactive. In the first configuration an external peripheral edge of the first active electrode distal-most surface may be entirely bounded by the second active electrode.

An example method of electrosurgically treating tissues along a patient airway is also disclosed herein, the method including positioning an electrosurgical wand in a first orientation so that a first active electrode treatment surface and a second active electrode treatment surface both engage a first target tissue along the patient airway, the first and second active electrodes arranged in an axially adjacent configuration. While in this first orientation and axially adjacent configuration, applying electrical energy between the first and second active electrodes and a return electrode of the electrosurgical wand to form, responsive to the energy, a localized plasma proximate to the first and second active electrode treatment surfaces and debulking, by the localized plasma, a portion of the first target tissue. Then axially adjusting the first active electrode treatment surface to be distally spaced from the second active electrode treatment, defining an axially offset configuration, and positioning the electrosurgical wand in a different orientation so that the first active electrode treatment surface is adjacent another target tissue along the patient airway. While the wand is in the second orientation and axially offset configuration, applying electrical energy between the first active electrode and a return electrode of the electrosurgical wand and forming, responsive to the energy, a localized plasma proximate to the first active electrode treatment surfaces and finely dissecting, by the localized plasma, a portion of the other target tissue.

In some example methods, the method may further comprise placing the first and second active electrodes in the axially adjacent configuration and applying electrical energy between the first and second active electrode, and the return electrode of the electrosurgical wand and coagulating, responsive to the energy applied, a portion of the tissues of the patient airway. In some methods, while finely dissecting, the second active electrode is dormant or electrically inactive. In the axially adjacent configuration, an external peripheral edge of the first active electrode treatment surface may be entirely bounded by the second active electrode treatment surface.

These and other features and advantages will be apparent from a reading of the following detailed description and a review of the associated drawings. It is to be understood that both the foregoing general description and the following detailed description are explanatory only and are not restrictive of aspects as claimed.

### NOTATION AND NOMENCLATURE

Certain terms are used throughout the following description and claims to refer to particular system components. As one skilled in the art will appreciate, companies that design and manufacture electrosurgical systems may refer to a component by different names. This document does not intend to distinguish between components that differ in name but not function.

In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections.

Reference to a singular item includes the possibility that there are plural of the same items present. More specifically, as used herein and in the appended claims, the singular forms “a,” “an,” “said” and “the” include plural references unless the context clearly dictates otherwise. It is further noted that the claims may be drafted to exclude any optional element. As such, this statement serves as antecedent basis for use of such exclusive terminology as “solely,” “only” and the like in connection with the recitation of claim elements, or use of a “negative” limitation. Lastly, it is to be appreciated that unless defined otherwise, all technical and scientific terms used herein have the same meaning as commonly understood by one of ordinary skill in the art to which this invention belongs.

“Ablation” shall mean removal of tissue based on tissue interaction with a plasma.

“Mode of ablation” shall refer to one or more characteristics of an ablation. Lack of ablation (i.e., a lack of plasma) shall not be considered an “ablation mode.” A mode which performs coagulation shall not be considered an “ablation mode.”

“Debulking” shall refer to removing tissue using ablation.

“Active electrode” shall mean an electrode of an electrosurgical wand which produces an electrically induced tissue-altering effect when brought into contact with, or close proximity to, a tissue targeted for treatment.

“Return electrode” shall mean an electrode of an electrosurgical wand which serves to provide a current flow path for electrical charges with respect to an active electrode, and/or an electrode of an electrical surgical wand which does not itself produce an electrically induced tissue-altering effect on tissue targeted for treatment.

Where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

### BRIEF DESCRIPTION OF THE DRAWINGS

The disclosure will be more fully understood by reference to the detailed description, in conjunction with the following figures, wherein:

FIG. 1 illustrates an electrosurgical system in accordance with this disclosure;

FIGS. 2 illustrate an example embodiment of an electrosurgical wand in accordance with this disclosure;

FIG. 3A illustrates a perspective view of an electrosurgical wand distal end in accordance with this disclosure;

FIG. 3B illustrates a top view thereof, perpendicular to an active electrode planar face of the electrosurgical wand in accordance with this disclosure;

FIG. 3C illustrates a side view thereof, in accordance with this disclosure;

FIG. 3D illustrates a perspective view of an underside thereof, in accordance with this disclosure;

FIG. 3E illustrates a partial cross section of the electrosurgical wand distal end, in accordance with this disclosure;

FIG. 4A illustrates a top view of the active electrode of the electrosurgical wand shown in FIG. 3A, in accordance with this disclosure;

FIG. 4B illustrates a cross section the active electrode thereof in accordance with this disclosure;

FIG. 5A illustrates a perspective view of an electrosurgical wand distal end in accordance with this disclosure;

FIG. 5B illustrates a side view thereof, in accordance with this disclosure;

FIG. 6A illustrates a perspective view of an electrosurgical wand distal end in accordance with this disclosure;

FIG. 6B illustrates a top view thereof, in accordance with this disclosure;

FIG. 7A illustrates a side view of an electrosurgical wand distal end in accordance with this disclosure;

FIG. 7B illustrates an end view of the electrosurgical wand distal end shown in FIG. 7A, absent the active electrode;

FIG. 7C illustrates an end view of the electrosurgical wand distal end shown in FIG. 7A, in accordance with this disclosure;

FIG. 7D illustrates the electrosurgical wand distal end shown in FIG. 7A, in a debulking configuration, in accordance with this disclosure;

FIG. 7E illustrates the electrosurgical wand distal end shown in FIG. 7A, in a second or fine dissecting configuration, in accordance with this disclosure;

FIG. 7C illustrates an end view of the electrosurgical wand distal end shown in FIG. 7A, in accordance with this disclosure;

FIGS. 8A and 8B illustrates various views of an electrosurgical wand distal end, the electrosurgical wand in a debulking configuration, in accordance with this disclosure; and

FIGS. 8C and 8D illustrates various views of the electrosurgical wand distal end shown in FIGS. 8A and 8B, the electrosurgical wand in a dissecting configuration, in accordance with this disclosure.

### DETAILED DESCRIPTION

In the description that follows, like components have been given the same reference numerals, regardless of whether they are shown in different examples. To illustrate example(s) in a clear and concise manner, the drawings may not necessarily be to scale and certain features may be shown in somewhat schematic form. Features that are described and/or illustrated with respect to one example may be used in the same way or in a similar way in one or more other examples and/or in combination with or instead of the features of the other examples.

As used in the specification and claims, for the purposes of describing and defining the invention, the terms “about” and “substantially” are used to represent the inherent degree of uncertainty that may be attributed to any quantitative comparison, value, measurement, or other representation. The terms “about” and “substantially” are also used herein to represent the degree by which a quantitative representation may vary from a stated reference without resulting in a change in the basic function of the subject matter at issue. “Comprise,” “include,” and/or plural forms of each are open ended and include the listed parts and can include additional parts that are not listed. “And/or” is open-ended and includes one or more of the listed parts and combinations of the listed parts. Use of the terms “upper,” “lower,” “upwards,” and the like is intended only to help in the clear description of the present disclosure and are not intended to limit the structure, positioning and/or operation of the disclosure in any manner.

Methods recited herein may be carried out in any order of the recited events, which is logically possible, as well as the recited order of events. Furthermore, where a range of values is provided, it is understood that every intervening value, between the upper and lower limit of that range and any other stated or intervening value in that stated range is encompassed within the invention. In addition, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Last, it is to be appreciated that unless defined otherwise, all technical and scientific terms used herein have the same meaning as commonly understood by one of ordinary skill in the art to which this invention belongs.

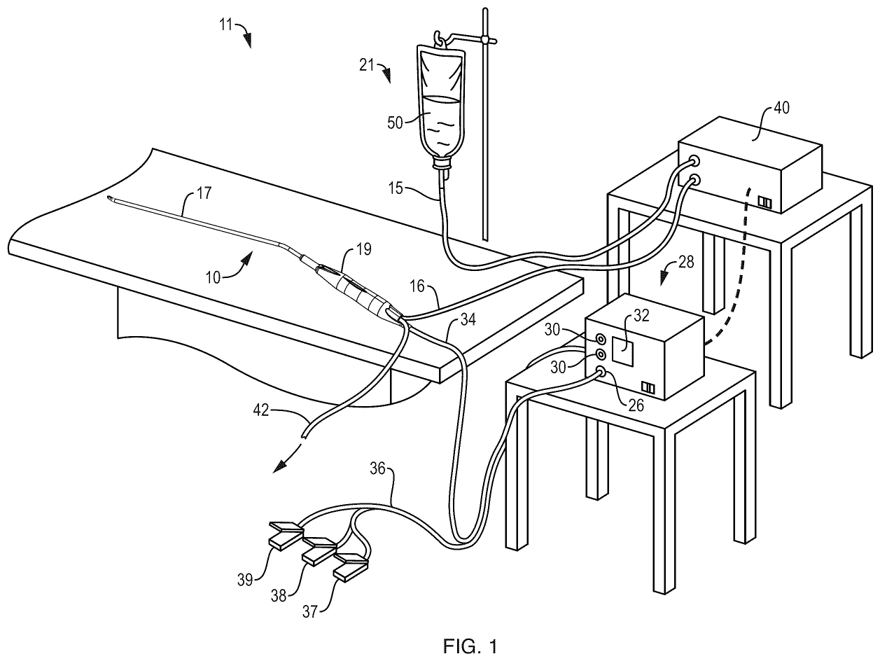

Referring to FIG. 1, an exemplary electrosurgical system 11 for treatment of tissue in accordance with the present disclosure will now be described in detail. Electrosurgical system 11 generally comprises electrosurgical wand 10, hereinafter “wand”, that may be electrically connected to an electrosurgical controller (i.e., power supply) 28, hereinafter “controller”; the controller 28 general configured to provide a high frequency voltage to the wand 10 and thereby to a target tissue site. The system may also include a fluid source 21 for supplying electrically conductive fluid 50 to wand 10 via fluid delivery tube 15/16. Fluid delivery may be controlled by pump 40, to provide a controlled fluid flow supply to wand 10 via delivery tube 16. Pump 40 may be in communication (shown as dotted line) with controller 28, such that selection of different electrosurgical power supply modes (described in detail later) may also communicate instructions to the pump 40, to alter a parameter of the pump 40 and adjust the fluid delivery rate. Pump 40 is shown as a separate enclosure but may be part of the same enclosure as the controller 28. In addition, electrosurgical system 11 may include a scope (not shown), that may include a fiber optic head light for viewing the surgical site, particularly useful in procedures in the back of the mouth. The scope may be integral with wand 10, or it may be a separate object. The scope may be a laryngoscope. The system 11 may also include a suction or aspiration tube 42 that may be configured to couple to a vacuum source (not shown), such as wall suction. Tube 42 as shown may be associated with the wand 10 for aspirating tissue debris and fluid from the target site. Tube 42 may also be operatively coupled to a pump (not shown) for example, such as a peristaltic pump, to control an aspiration flow rate.

Wand 10 generally comprises a handle 19 and an elongate tubular shaft 17 extending distally from handle 19. The handle 19 typically comprises a plastic material that is easily molded into a suitable shape for handling by the surgeon. As shown, a connecting cable 34 has a connector 26, and together they electrically couple the wand 10 to controller 28. Controller 28 may have an operator controllable energy/voltage level adjustment 30 to change the applied voltage level, which is observable at a display 32. Controller 28 may also include first second- and third-foot pedals 37, 38, 39 and a cable 36, which may be removably operatively coupled to controller 28. The foot pedals 37, 38, 39 may allow the surgeon to remotely adjust the voltage, mode or energy level applied to active electrode. In an exemplary embodiment, first foot pedal 37 may be used to direct the controller 28 to deliver energy to the wand 10 in the “ablation” mode and second foot pedal 38 may place electrosurgical controller 28 into a thermally heating mode (i.e., contraction, coagulation, or other types of tissue modification without volumetric tissue removal/debulking). Alternatively, second foot pedal may direct the electrosurgical controller to supply energy in a “blended” mode (blend of tissue removal or debulking and concomitant hemostasis). The third foot pedal 39 (or in some embodiments a foot-activated button) may allow the user to adjust the voltage level within the mode. In other embodiments, a series of hand switches along the wand handle 19 may replace at least some of the foot pedals.

The electrosurgical system 11 of the various embodiments may have a variety of operational modes. One such mode may employ Coblation® technology. The assignee of the present invention owns and developed Coblation® technology. A more detailed description of this technology can be found in commonly assigned U.S. Pat. No. 5,697,882, 6,355,032; 6,149,120 and 6,296,136, the complete disclosure of which is incorporated herein by reference. The electrosurgical system 11 may include a blended mode wherein a blend of tissue debulking and thermal shrinkage may occur within the same mode. A more detailed description of this mode can be found in commonly assigned U.S. Pat. No. 11,116,569, the complete disclosure of which is incorporated herein by reference. The electrosurgical system 11 may include a pulsing thermal mode wherein the tissue is thermally treated to coagulate and shrink the turbinate tissue, pulsing intermittently with an ablation output, wherein the ionized vapor formed may be configured to reduce tissue sticking.

In the thermal heating or shrinking (coagulation) mode, the controller 28 mat apply a sufficiently low voltage to the active electrode to avoid vaporization of the electrically conductive fluid and subsequent molecular dissociation of the tissue. The surgeon may automatically toggle the controller 28 between the ablation and thermal heating modes, by alternatively stepping on foot pedals 37, 38, respectively. This allows, for example, the surgeon to quickly move between coagulation and ablation in situ, without having to remove his/her concentration from the surgical field or without having to request an assistant to switch the controller. By way of example, as the surgeon sculpts or dissects soft tissue in the ablation mode, the wand typically may simultaneously seal and/or coagulation small, severed vessels within the tissue. However, larger vessels, or vessels with high fluid pressures (e.g., arterial vessels) may not be sealed in the ablation mode. Accordingly, the surgeon can simply step on foot pedal 38, automatically lowering the voltage level below the threshold level for ablation and apply sufficient pressure onto the severed vessel for a sufficient period to seal and/or coagulate the vessel. After this is completed, the surgeon may quickly move back into the ablation mode by stepping on foot pedal 37. By way of a second example, the surgeon may finely dissect nodes or polyps along the patient airway via an ablation mode and then coagulate any bleeders with a coagulation mode. In another example, the surgeon may fine dissect a portion of the vocal cord using a higher voltage Ablation mode during a Cordectomy and then reduce the voltage or chose a “coag” mode, to coagulate any resulting bleeders. In some procedures, the surgeon may select a high voltage ablation mode to debulk inflamed or scar tissue along the Subglottis to treat subglottic stenosis. Selecting each mode may also automatically adjust a fluid delivery rate to the wand distal end. For example, selecting the debulking mode(s) may also direct the controller 28 to operate the pump 40 to deliver fluid at a rate configured to support the target rate of debulking the tissue, and selecting a thermal heating mode may direct the controller 28 to operate the pump 40 to deliver fluid at a rate figured to support thermally heating the tissue. The fluid delivery flow rate for debulking may be higher than the fluid delivery flow rate for thermally heating.

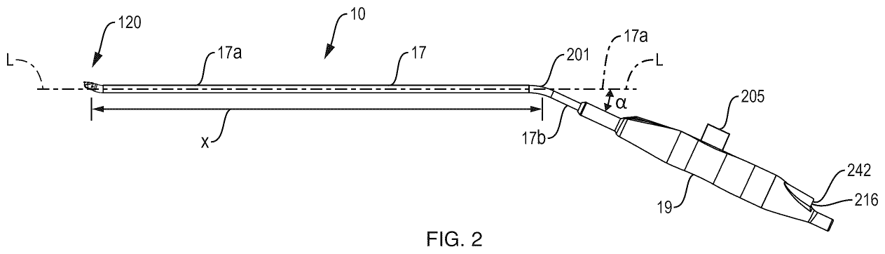

FIG. 2 illustrates a side view of electrosurgical wand 10 constructed according to the principles of the present disclosure and configured to operate with system 11. As shown in FIG. 2, wand 10 generally includes an elongate shaft 17 and a handle 19 coupled to the proximal end of shaft 17. Handle 19 typically comprises a plastic material that is easily molded into a suitable shape for handling by the surgeon. Handle 19 defines an inner cavity that may house electrical cabling and connections (not shown). Housing may provide a suitable interface for connection to an electrical connecting cable, such as cable 34. Inner cavity may also house fluid conduits for aspiration and fluid delivery.

Fluid inlet 216 may form a part of the fluid delivery conduit of the whole system, fluid delivery conduit defining a construct that is configured to deliver electrically conductive fluid 50 from the source 21 to the wand distal portion 120. Fluid inlet 216 may be fluidly coupled tube 16. Fluid inlet 216 may be fluidly coupled by an operator to a tube 16, that may be provided separated from handle 19 and fluid supply source 50. In other example embodiments wand 10 may be provided with fluid delivery tube 16 pre-attached such that tube 16 may extend through inlet 216; inlet 216 defining an opening through the handle 19 for receiving tube 16 therethrough. Fluid delivery conduit may extend through handle 19 (not shown) and along shaft 17. Fluid delivery conduit may be defined by an inner bore surface of shaft 17. In some embodiments, wand 10 may also include a valve or equivalent structure (not shown) on the wand 10 or tubing 16, for controlling the flow rate of the electrically conducting fluid delivered to the target site. In other embodiments, as disclosed herein the flow rate may be controlled by pump 40.

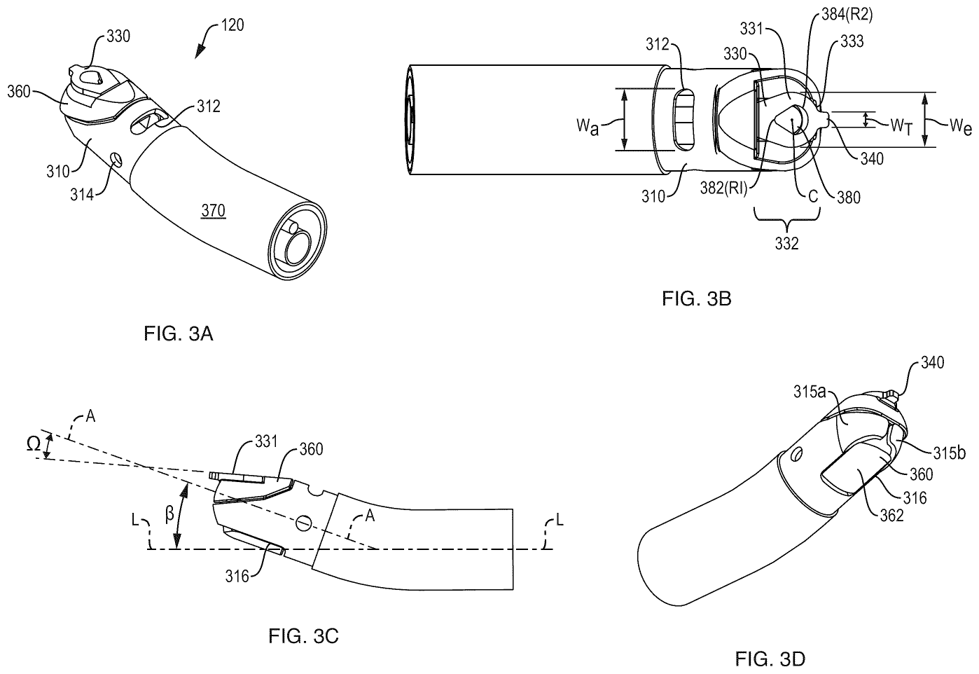

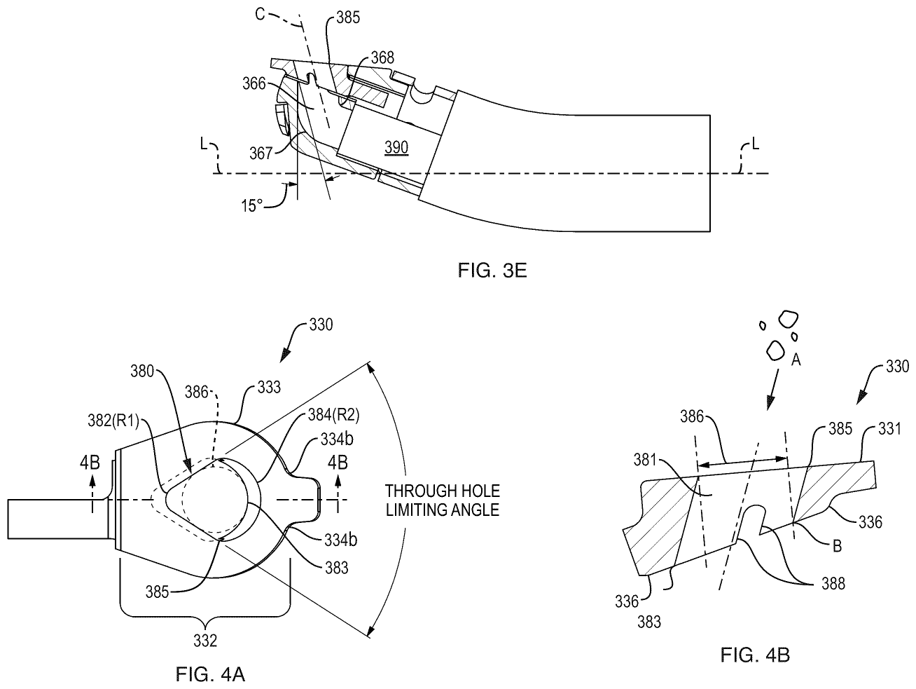

A fluid aspiration conduit may also extend through an opening 242 in handle 19, fluid aspiration conduit defining a construct configured to remove fluid from the wand distal end 120 and away from the treatment site. Fluid aspiration conduit may extend from wand distal working end 120, to remove fluid and debris therefrom. Fluid aspiration conduit may be fluidly coupled to or selectively coupled to tubing 42 that may couple to a vacuum source. Fluid aspiration conduit may include a tube 390 (shown in FIG. 3E) that extends from the wand distal working end 120 proximally along the shaft 17 and handle 19 up to tube 42. Tube 390 and tube 42 may different length portions of the same single component. Aspiration may be controlled manually via a switch 205 on the handle 19 that is in communication with a valve (either mechanically or electrically). In other embodiments aspiration may be automatically controlled via controller 28 and may automatically initiate or adjust a valve while energy is being delivered to the wand distal working end 120.

Wand 10 is generally configured to improved access to tissues within a patient's airway that may be adjacent the larynx, and therefore shaft 17 may include a bend or curve 201. Curve 201 may be closer to the handle 19 than distal working end 120. If we allocate the shaft to include a distal segment 17a and a proximal segment 17b, as shown, bend 201 may angularly offset the proximal shaft segment 17b at an angle α between 30-55 angular degrees from a longitudinal axis (L-L) of the shaft distal segment 17a. This angular offset may improve access along the patient airway and visualization of the target area. More preferably the angle α may be approximately 35 degrees, as the inventors have found that this shallow angle may more precisely control the distal working end 120, while allowing some visibility of the projecting distal tip of active electrode (discussed in more detail hereinafter). Shaft distal segment 17a may be a working length (X) that extends through an inner opening of a laryngoscope and is sufficiently long to gain access to the target area, and may be at least 17 cm long, as measured from an apex of the bend 201. In some preferred embodiments, the distal segment 17a may be approximately 25 cm long, as this may improve subglottic access. Shaft 17 may be formed of annealed steel, to add elastic flexibility to the shaft for improved manipulation along the patient airway.

FIGS. 3A-3E show a first embodiment distal working end 120 of wand 10. Distal working end 120 may have a bipolar arrangement and include a return electrode 310 and an active electrode 330. An electrically insulative spacer 360 (hereinafter “spacer”) may support the active electrode 330 and electrically isolate the return electrode 310 from the active electrode 330. Spacer 360 may be formed of a plasma resistant ceramic and may also define a portion of the back side of distal end 120 (best seen in FIGS. 3C and 3D) to thermally isolate this side, as discussed in more detail, hereinafter. In general, the distal working end 120 left side is a mirror image of the right side, and therefore features shown on one side such as aspiration holes and notches are not specifically shown in a figure but are inherently present. Distal working end 120 may be configured to treat tissue via plasma generation around the active electrode 330 which may therefore be formed of material or materials that are resistant to plasma degradation. Example materials include, but are not limited to, tungsten, titanium, molybdenum, stainless steel, aluminum, gold, copper or the like. Active electrode 330 may be a complex unibody with a variety of edges of surfaces, some of which are intended to control the tissue effect, and of the edges and surfaces are intended to aid in resisting or mitigating clogging of the wand. In general, the size of the active electrode 330 overall is minimized and thereby requiring a minimal amount of energy to treat the delicate structures along a patient's airway. Being small in size also helps confine the overall profile of the wand distal end 120. The active electrode 330 may be formed as a single molded body.

Return electrode 310 may be a tubular shaped conductive material, that may be an extension of and exposed portion of shaft 17. Return electrode 310 may be formed on annealed stainless steel. Most of the shaft 17 may be covered with insulating shrink tubing 370 to limit the return electrode exposed surface area and avoid inadvertent tissue damage along the patient airway, proximal of the distal working end 120. Return electrode 310 may include apertures 312, 314 therethrough that may be in fluid communication with the fluid delivery conduit, the conduit extending within the shaft 17 and coupled to tubing 16, as explained previously. A portion of the fluid delivery conduit may define a boundary formed by an inner surface of the shaft 17, or alternatively may include tubing (not shown) that extends along the shaft 17. Apertures 312, 314 may therefore function as fluid delivery apertures for delivering an electrically conductive fluid 50 to external surfaces of the distal working end 120. Aperture 312 may define an elongate 360-degree bounded hole with a longer dimension that extends circumferentially around the tubular return electrode 310. Aperture 312 may be centered relative to a longitudinal axis of the working end 120. Aperture 312 may define a maximum length dimension (Wₐ) that is greater than a corresponding maximum width dimension (Wₑ) of the active electrode 330, best seen in FIG. 3B. Fluid dispensed from aperture 312 may generally be drawn distally, towards the active electrode 330 because of gravity and also aspiration through aspiration opening 380 (described in more detail later).

Return electrode bilateral apertures 314 (only one shown), may supplement the fluid 50 delivered through aperture 312 and serve to increase a wetted surface area of the return electrode exposed surface. Treatment along a patient airway is generally considered a dry environment, relative to the fluid filled enclosed cavities such as during arthroscopic surgery, for example. Multiple spaced apart fluid delivery locations, such as through apertures 312 and 314 provides an environment wherein electrically conductive fluid 50 wets a larger surface area of the return electrode 310 and extends around the active electrode perimeter further. This provides an improved environment for uniform plasma formation.

Return electrode 310 may also include an aperture 316 (best seen in FIG. 3D) on the wand under side, to expose the spacer 360. This may reduce a thermal footprint of the return electrode 310 and may limit inadvertent thermal damage should adjacent tissue touch the back side of the wand distal end 120. Spacer 360 may be formed or coated with a ceramic that is a material that may act as a heat sink. Spacer 360 may include a radial projection 362 that extends through aperture 316 up to at least the circumferential outer-most surface of return 310, to provide a smooth continuous outer-most back surface. This may reduce snagging and provide a preferable contact surface to engage the adjacent tissues on the back side of the wand distal end 120, which may reduce inadvertent contact between this tissue and the return electrode 310.

Aperture 316 may be bounded by bilateral arms 315a, 315b of return electrode 310, and each arm 315a, 315b may encircle a distal-most surface of spacer 360. Aperture 316 may be formed by obtaining the return electrode 310 with the bilateral arms 315a, 315b in a substantially straight or spaced apart orientation, configured to receive the spacer 360 therebetween. Spacer 360 may then be assembled and placed between the bilateral arms 315a, 315b, before plastically deforming the bilateral arms 315a, 315b towards each other and around a distal most surface of spacer 360. Return electrode 310 may therefore be formed of a readily plastically formable electrically conductive material, such as annealed stainless steel. Wrapping these arms 315a, 315bmay place a portion of the return electrode 310 close the distal tip (340) of active electrode 330, while maintaining a small distal end wand profile. In other embodiments, aperture 316 may be provided as a 360-degree bounded hole, preformed and the spacer 360 may snap into place. However, in order to assemble in this fashion, inventors have found that the distal end profile of the wand 10 may need to be larger to enable this assembly, and therefore may be less preferable.

Having a portion of the return electrode (such as arms 315a, 315b) wrapped around a distal-most surface of spacer and directly adjacent distally projecting tip 340 of active electrode 330 (without making electrical contact) may help to initiate a rapid and uniform vapor layer and ultimately speed up plasma initiation at the projecting tip 340. Having the return electrode 310 directly under the projecting tip 340 may provide increased energy density directly between the active electrode projecting tip 340 and return electrode 310, helping to initiate more rapid plasma formation at the distal projecting tip 340. Having a proximity between the projecting tip 340 and return electrode 310 (and more specifically arms 315a, 315b) also eases the burden for sufficient electrically conductive fluid between the electrodes. This burden stems from having to flow sufficient electrically conductive fluid from the delivery apertures 312, 314 that are proximal of the entire active electrode 330 all the way around to this distal facing surface and near the tip 340, which can be frustrated depending on a variety of factors. For example, fluid 50 may be drawn into the aspiration opening 380 through the active electrode 330 before reaching this distal most surface, or this fluid 50 may fall away from the wand 10, depending on the orientation of the wand 10. Secondly, moving sufficient fluid from the delivery apertures 312, 314 that are proximal of the entire active electrode 330 all the way around to this distal facing surface may take time, causing a frustrating time delay between actuating the fluid delivery and energy and the fluid reaching around to this distal facing surface and near the tip 340. As a reminder this fluid 50 is key to enabling plasma formation. Therefore, placing a return electrode 310 directly under and close to the active electrode tip 340 may ease the burden on supplying sufficient electrically conductive fluid in a reasonable time, to electrically bridge the two electrodes, required for forming uniform or consistent plasma. This reduced burden helps initiate a vapor layer more immediately after energy and fluid delivery actuation, ultimately providing plasma initiation in a reasonable time.

In addition, having the return electrode 310 wrap around this distal end, enables the return electrode 310 to have direct contact with tissue closer to the target tissue. This forms a more concentrated and more uniform electric field around the distal radius, so when the wand distal surfaces touch tissue, there is less distance for some of the electrical current to travel through tissue. This results in more tissue cutting with ablation and less resistive heating of the tissue. This provides for fine tissue dissection by molecular dissociation, with reduced thermal spread. This is important for laryngeal applications, to limit inadvertent thermal damage to surrounding delicate airway anatomy.

FIG. 3C shows a left side view of distal working end 120. Distal working end 120 may have a longitudinal axis A-A that is angularly offset by angle β from shaft longitudinal axis L-L. Angle β may be between 5-30 degrees and may more preferably be approximately 20 degrees to enable visibility of the target tissues within the patient airway, while also fitting within a laryngoscope opening. Active electrode 330 may define a top planar surface 331 that extends an angle Ω relative to working end longitudinal axis A-A. Angle Ω may be between 5 and 10 degrees. Angle Ω is configured to allow the operator to see the projecting tip 340 of the active electrode while treating tissues within the patient airway. Angle βmay angularly offset the distal working end 120 and thereby active electrode planar surface 311 in a first direction relative to the longitudinal axis L-L and angle Ω may angularly offset the active electrode planar surface in a second direction, relative to the longitudinal axis L-L, the second direction opposite the first direction.

FIG. 3B shows a top view of distal working end 120, perpendicular to active electrode planar top surface 331. Active electrode top surface 331 may be planar along its entire length (best seen in FIG. 3C). During use, this planar surface 331 may be placed on the target tissue and upon application of electrosurgical energy, may debulk this target tissue. Active electrode 330 includes a proximal annular portion 332, that includes a 360-degree bounded aspiration opening 380 therethrough. Annular portion 332 may be axially coextensive with spacer 360. A projecting tip 340 extends distally from the annular portion 332. The projecting tip 340 has a top planar surface that forms a portion of the active electrode top surface 331. Stated another way, projecting tip 340 has a top planar surface continuous with and coplanar with annular portion top planar surface. Projecting tip 340 may extend axially beyond spacer distal-most surface and be unsupported by spacer 360. Projecting tip 340 may have a length between 0.010-0.065 inches. Projecting tip 340 is generally configured to finely dissect a target tissue via a formation of plasma therealong and having the tip 340 project beyond the spacer 360 provides surfaces on a plurality of sides (up to 5 sides) of the projecting tip 340 that may better access and treat this target tissue.

Active electrode 330 peripheral edge boundary 333 may include bilateral concave curves 334a, 334b (FIG. 4A) that define the transition from the annular portion 332 and projecting tip 340. Projecting tip 340 may have a maximum lateral width W_{T} of between 0.020-0.025 inches, while a maximum lateral width Wₐ of the annular portion may be between 0.070-0.090 inches. A ratio of the maximum lateral width Wₐ to W_{T} may be at least 2:1 and may more preferably be at least 3:1. This may provide sufficient electrode planar surface 331 to debulk tissue, with a sufficiently narrow projecting tip 340 to finely dissect a target tissue. Maximum lateral width W_{T} may be less than a corresponding maximal opening size Wₑ of aspiration opening 380, which may be between 0.035-0.045 inches.

Aspiration opening 380 is configured to aspirate plasma by-products, partially digested tissue and fluid therethrough, and is fluidly coupled to the fluid aspiration conduit of the system 11. More specifically, aspiration opening 380 may be in direct fluid communication with an aspiration cavity 366 within the spacer 360, which is in direct fluid communication with a suction tube 390 that extends along the shaft 17. Aspiration opening 380 defines a complex opening that extends from the top planar surface 331 to a bottom surface 336 of active electrode 330, the opening 380 including several structural features that provide a sufficiently large opening for efficient aspiration of plasma by-products and partially digested tissue therethrough while reducing the likelihood of clogging with the partially digested tissue, along the aspiration conduit. Too large an aspiration opening may allow larger size tails or strings of partially digested tissue into the wand 10, that may clog the aspiration conduit. In addition, too large an aspiration opening has been found to form a central untreated core of tissue. However, too small an aspiration opening may limit aspiration completely, and leave the plasma by-products and partially digested tissue in the field. This complex opening is configured to provide sufficient opening size for effective aspiration, while managing the aspirated plasma by-products and partially digested tissue to avoid clogging.

At least some of the means of managing the aspirated tissue to mitigate clogging includes means to further digest the partially digested tissue. This means is best described while viewing FIGS. 4A and 4B. Consider first that as partially digested tissue enters the opening 380, some of this tissue may further interact with plasma formed along the inner surface 381 and recessed edges of the opening 380 as this tissue flows along the opening 380. However, at a certain distance away from this inner surface 381, central to the opening 380, the partially digested tissue may not interact with any plasma. This forms or defines a plasma-remote zone 386 along the opening 380, that is remote from the inner surface 381 and thereby less effected by the plasma. Tissue debris that may include plasma by-products within the plasma-remote zone 386 may be remote for further digestion as it is aspirated through the opening. Stated another way, as tissue debris flows through the opening 380 spaced away from inner surfaces 381, there ends up with a central zone 386 or tissue debris, that may be cylindrical, that may not be digested further via plasma.

A first means to diminish this zone 386 and further digest this tissue is provided via the angle of the aspiration opening inner surface 381 (or boundary walls). These inner surfaces 381 may extend through the active electrode 330 defining a constant cross section therethrough, extending along a central axis (C) that extends at an incline relative to the planar top surface 331. The central axis C may extend proximally at approximately 20 degrees (°) from an axis perpendicular to the top planar surface 331. The incline angle helps to increase the effective length of the inner surfaces 381, and thereby increase a length available for further debris digestion via interacting with the plasma formed therealong. In addition, the angled opening sets up a 360-degree edge boundary 385 on the top planar surface 331 that is axially offset from a corresponding 360-degree edge boundary 383 on a corresponding bottom surface of the active electrode 330. The central axis C (and thereby the aspiration opening walls) may incline so to extend proximally as the opening 380 extends through the active electrode 330 and away from the top planar surface 331, so that the edge boundary 383 on the bottom surface is proximally offset from the top surface edge boundary 385. This axial offset helps to reduce the effective diameter (or size) of zone 386, as the flow of tissue and debris is at least partially interrupted by the bottom surface edge boundary 383. The incline angle is configured to provide a larger overall opening dimension while limiting the zone 386.

Aspiration opening 380 may be non-circular and may be shaped like a rounded wedge. Aspiration opening 380 may define a 360-degree bounded hole with a proximal-most curve 382 with a first radius of curvature that may be between 0.006-0.010 inches (R1) two bilateral linear edges that extend angularly and distally from the proximal-most curve up to a distal-most curve 384 (R2) with a second radius of curvature that may be between 0.015-0.025 inches. In some embodiments, the ratio of R2 to R1 may be at least 3:1. In some embodiments the two bilateral linear edges may extend at least a 60 degree (angular) relative to each other. This aspiration opening shape provides a large opening (as defined by distal-most curve 384) sufficient to remove sufficient tissue debris therethrough. The larger radius of curvature (R2) provides an increased opening size, while also providing a larger surface area for further digestion via plasma along this distal most segment inner surface (381), as the aspirating tissue flows along the aspiration opening. The narrower apex 382 however limits a size of a proximal side of the zone 386, and therefore reduces zone cross section size 386.

In addition, notches 388 are forms along the bottom surface edge boundary 383 to further digest the aspirating partially digested tissue within the zone 386. Plasma preferentially forms and may be stronger and extend further away along asperities on the active electrode 330. Bilateral notches 388 are therefore formed along the bottom surface boundary 383, the notches 388 axially coincident with the two bilateral linear edges of the aspiration opening cross section, that is coextensive with a wider portion of the zone 386.

Tissue debris therefore enter the aspiration opening 380 at an angle approximately perpendicular to the top planar face 331, illustrated as arrow A. Therefore, as tissue debris enters the opening 380, it may be first digested at edge boundary 385, including apex 382 and bilateral linear edges. Furthermore, tissue debris may be further digested as it interacts with plasma formed along the inner surface 381, including the large inclined distal portion of inner surface. The inclined distal surface (angle β) may increase the length of contact and thereby plasma interaction to improve tissue digestion. Lastly the partially digested tissue may interact with bilateral notches 388 to further digest it before it enters the spacer cavity 366.

This inclined angle β may also deflect the flow of tissue debris though the aspiration opening 380 away from a distal most wall 367 of the spacer cavity 366, to avoid the tissue debris from collecting there. This inclined angle β directs flow of tissue debris towards the suction tube 390.

FIG. 3E shows a side view of the distal end 120, with a portion removed to reveal a cross section of the active electrode 330, spacer 360 and return electrode 310, as well as other component. FIG. 3E illustrates a curved aspiration cavity 366 within spacer 360, that forms a part of the aspiration conduit. Aspiration cavity 366 is in fluid communication between aspiration opening 380 and suction tubing 390. Curved aspiration conduits tend to be conducive to tissue clogging the conduit, therefore corner 368 may be rounded to increases the flow rate around this inner corner of the curve to help prevent clogging when moving debris around the corner.

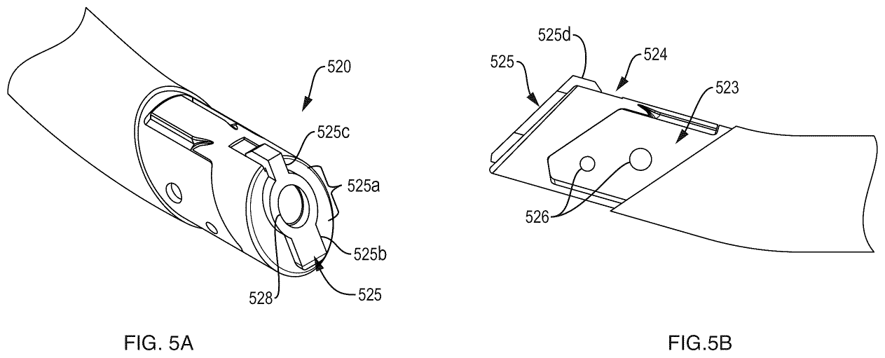

FIG. 5A-5B illustrates another other example embodiment distal working end of wand 10, in accordance with this disclosure. FIG. 5A illustrates a distal end 520 of an electrosurgical wand 10, with an active electrode 525 that defines a distal-most surface of the wand 10. Active electrode 525 defines a planar distal facing surface that includes an annular portion 525a with a first leg 525b extending from a first side of annular portion 525a and a second leg 525c extending from a second opposing side of the annular portion 525a. First leg 525b may extend distally from annular portion 525a up to a distal most edge end thereof. First leg 525b may terminate proximally spaced from a spacer distal most surface. Second leg 525c may also wrap around a distal edge surface of spacer 524 and extend proximally along the working distal end 520 and provide finer tissue dissection in that region. Second leg 525c may wrap around the distal edge surface and form a projection, such as a triangular or toothed projection 525d, extending radially away from spacer surface, to improve fine dissection of the target tissue. Toothed projection 525d may extend proximally along wand distal end 520. Active electrode 525 may be generally oblong, having a length larger than a maximum width, the maximum width perpendicular to the length. Similar to previous embodiments, return electrode 523 may be perforated with a plurality of fluid delivery ports 526 provide electrically conductive fluid and bridge the active and return electrode 523, to generate plasma.

Annular portion 525a may include a 360-degree bounded hole that serves as an aspiration opening 528, similar to other embodiments disclosed herein. Annular portion 525a may define the largest lateral width portion of the active electrode 525 and may be at least 50% greater than a maximum corresponding width of the remainder of the active electrode (525b, 525c, 525d).

In use, the distal facing planar surface of active electrode 525 may engage a target tissue to debulk the tissue, while supplying energy configured to ablating tissue and remove the ablated tissue through aspiration opening 528. For finer dissection, the second leg 525c and projection 525d may preferably engage the target tissue with the distal facing planar surface spaced away from the target tissue for dissection.

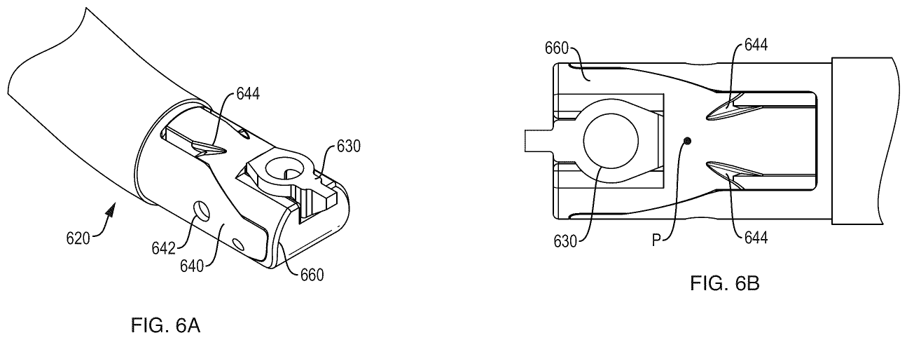

FIG. 6A and 6B illustrates another example embodiment of a distal working end of wand 10 including an active electrode 630, a return electrode 640 and a spacer 660. Return electrode 640 may include a plurality of apertures 642 therethrough in fluid communication with the fluid delivery conduit, the conduit in fluid communication with an electrically conductive fluid source 50, as disclosed herein. In addition, spacer 660 may form bilateral tear ducts (directive saline ports) 644 in fluid communication with fluid delivery conduit and source 50. Ducts 644 may direct fluid delivery across the external surface of the spacer 660 and towards a midline of the wand distal working end 620. Tear ducts 644 are shallow and may help retain the electrically conductive fluid 50 in contact with spacer 660 for any orientation of the wand. In use wand 10 may be turned upside down to target tissue, and fluid 50 on the wand surface may tend to quickly fall away from the distal working end 620. Maintaining wetted surfaces around the working distal end improves plasma generation and consistency. The ducts 644 are configured to direct small amounts of electrically conductive fluid 50 along the working distal end 620, maintaining contact and resisting separation therefrom despite the wand orientation. Each duct 644 may define tapering channels along an external surface of spacer 660, tapered both in depth and width as they extend distally. Each duct 644 may extend along a duct axis that is angled relative to the distal working end longitudinal axis. Each duct axis, in projection, may intersect with each other at a center of the distal working end at a point (P) that may be proximally spaced from active electrode 630. Fluid delivery path may include flowing fluid along ducts 644 in a general distal direction, where they are aimed (by axis of ducts) to combine towards the center of the top surface of spacer 660 and then flow generally axially and distally towards aspiration opening. Ducts 644 may be in fluid communication with the fluid delivery conduit that is also in fluid communication with apertures 642.

### Moveable Electrode

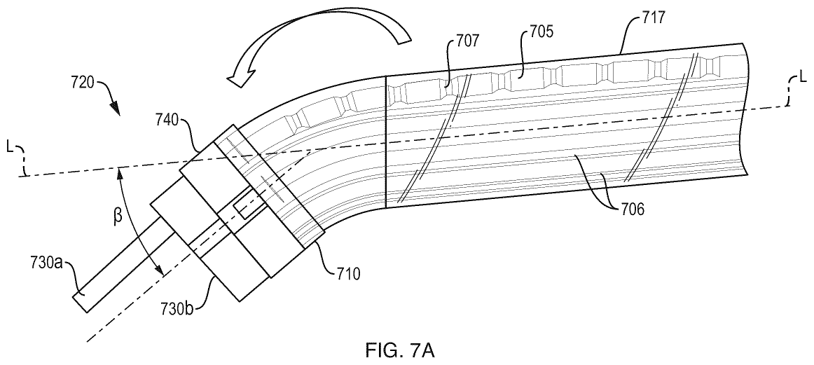

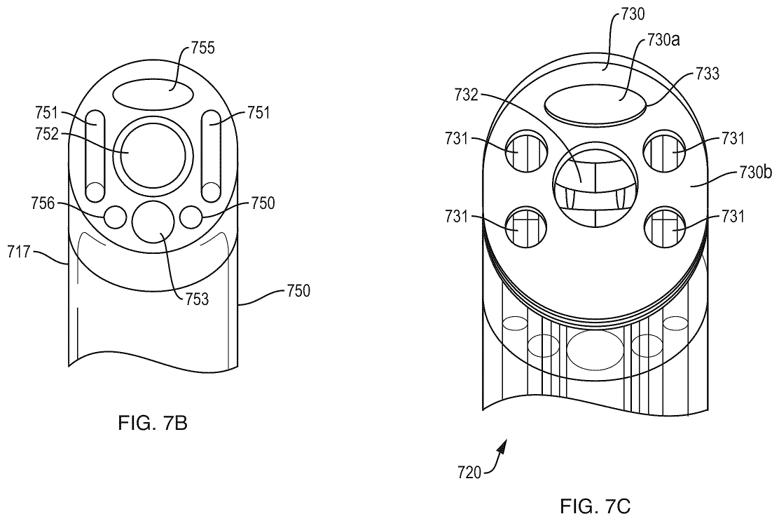

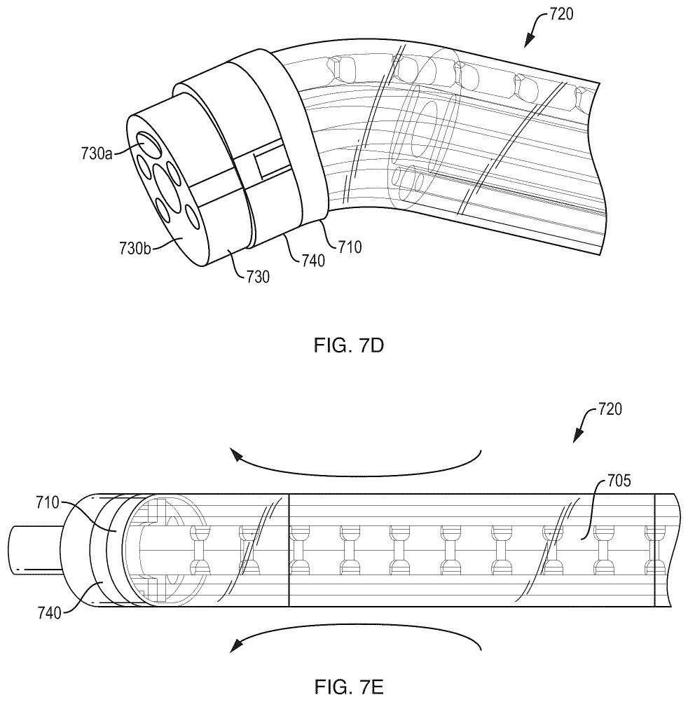

FIG. 7A-7E illustrate an alternative embodiment of an electrosurgical wand distal end 720 that may be configured to electrosurgically treat tissue along a patient airway. Wand distal end 720 may couple to controller 28 and include an aspiration conduit that may fluidly couple to tubing 42, and a fluid delivery conduit that may fluidly couple to tubing 16 (FIG. 1). Wand distal end 720 may include electrodes in a bipolar arrangement, including a return electrode 710, a combination active electrode 730 that may include a first active electrode portion 730a and a second active electrode portion 730b, and a spacer 740 therebetween. The first active electrode portion (730a) may be axially moveable between a first and second configuration. In the first configuration, the combination wand may be configured to debulk tissue and the first and second portion may define a single continuous treatment surface that may lie substantially on the same plane. In the second configuration the first active electrode portion may be moved away from the remaining portion of the combination electrode, and this first portion 730a may be used to finely dissect a target tissue.

Starting with FIG. 7A, wand working distal end 720 may be angularly offset from the shaft by angle β. In this embodiment, the angular offset β may be adjustable via at least one articulating shaft. Articulation may be provided by two tensioning wires 706 along an inner radius of the angular offset, wires 706 placed in tension to articulate working distal end 720. Spine 705 may be configured to provide stiffness to the device and yet flex as the wand articulates. Spine 705 may be passive in terms of articulation, and articulation may be provided solely via wires 706. In some embodiments, the spine 705 may include a plurality of cuts 707 that are sized and shaped to enable flexing of the shaft 705 during articulation. Wires 706 and spine 705 may extend along shaft 717 and couple to actuation means associated with the handle 19, using means that is known in the art. Shaft 717 may include different material therealong to adjust the bending force and may include shape memory materials. Spine 705 and Wires 706 may be operatively coupled to a variety of actuation means such as a combination of triggers, thumb pushers (for the retraction or extension of the micro shaft), buttons, or knobs, which may all be located along handle 19.

Shaft 717 may include a multi-lumen extruded tube 750, shown separated from the remains of the wand 72 in FIG. 7B, for ease of understanding. Multi lumen tube 750 may be formed with flexible PVC and or silicone. Each lumen of the multi-lumen extruded tube 750 may provide a different function to the working distal end 720, such as at least one fluid and or drug delivery conduit 751, at least one fluid aspiration conduit 752, at least one electrically conductive wire conduit 753, conduits 756 for the wires 706 and the spine conduit 755. FIG. 7C illustrates the multi-lumen extruded tube 750 with the active electrode 730 assembled thereto. Active electrode 730 may include a first active electrode 730a and a second active electrode 730b, and second active electrode 730b may be a stationary electrode with a plurality of apertures therethrough. These may include fluid delivery apertures 731 that are in fluid communication with fluid and or drug delivery conduit 751. These may include a central aperture 732 that may be in fluid communication with aspiration conduit 752. Second active electrode 730b may include an aperture 733 to receive first active electrode 730a therethrough. Aperture 733 may define a 360-degree bounded hole. Aperture 733 may define an elongate or oval shape that slidingly receives first active electrode 730a therethrough, that may be substantially the same shape as bounded hole.

First active electrode 730a may define a distal exposed end of a spine 705. First active electrode 730a may be axially moveable to define an axial offset between the two active electrode (730a, 730b) to at least partially define the tissue effect mode. Axially moving the first active electrode 730a may change the surface area and edge surfaces available to treat tissue, which in combination with different energy outputs from controller 28, may offer differing treatment modes. The first and second active electrodes (730a, 730b) may make up the entire combination active electrode 730 in total. In other embodiments, there may be third and fourth active electrodes, that may all be independently axially moveable to alter the mode of tissue treatment.

In use, the combination active electrode 730 may have a first configuration, wherein distal most treatment surfaces of the first and second active electrodes (730a, 730b) may be coextensive with each other. As shown in FIGS. 7D, these distal most treatment surfaces may be coplanar with each other, to define a continuous single distal facing planar surface of the active electrode 730. In the first configuration, the active electrode 730 is configured to debulk the target tissue, by engaging the distal surfaces of both the first and second active electrode (730a, 730b) with a target tissue. Energy supplied from the controller 28 may be delivered to both active electrodes (730a, 730b) and may be configured to debulk the target tissue.

In a second configuration, the first active electrode 730a may be axially offset (along the longitudinal axis of the wand distal end 720). Stated another way, by way of example, if the angular offset β of the distal end 720 from the shaft longitudinal axis L-L is 30 degrees, then the first active electrode 730a may also axially extend along an axis approximately 30 degrees relative to the longitudinal axis L-L. The first active electrode 730a may now provide a focused treatment electrode configured to finely dissect the target tissue or tissues adjacent thereto. The first active electrode 730a may define a terminal end of wand spine 705 that extends along the wand shaft within one of the preformed lumens. The first active electrode 730a is preferably moved to an axially advanced location (second configuration) and then energy supplied to the wand distal end, while the first active electrode 730a remains stationary. Then, upon the operator wishing to debulk tissue, the first active electrode 730a is retracted to the first configuration by the operator and energy supplied to the wand distal end to debulk tissue, which the first active electrode 730a remains in the first configuration.

First active electrode 730a may be substantially smaller in cross section than the second active electrode 703b. The first and second active electrode 730a, 730b may be electrically isolated from each other, such that only the first active electrode or the second active electrode may be selectively coupled to the energy supply. When the first active electrode 730a is axially advanced towards the second configuration, for finer dissection, the second active electrode 730b may be dormant or in other words non-active or incapable of electrosurgically treating tissue. Spine 705 may be formed of an electrically conductive material to provide the electrical connection to first active electrode 730a but may be coated or covered along its length to prevent electrically communication between the first active electrode 730a and the second 730b.

First active electrode 730a may be advanced axially along the longitudinal axis of the distal end 720 relative to the second portion 730b. The first active electrode 730a may have a boundary or periphery that may be entirely surrounded by the second active electrode 730b, when in the first configuration. Stated in another way, in the first configuration, the first active electrode 730a may be entirely surrounded by the second active electrode 730b and the second active electrode 730b may define the entire combination electrode 730 outer-most periphery. In other example configurations, the first active electrode 730a when in the first configuration may have a boundary or periphery that defines a portion of the boundary of the whole combination active electrode 730 (as shown in later embodiments). The first active electrode 730a may define a center that is offset from a combination electrode center and may be disposed towards an outer side of the active electrode, the outer side being defined as the outer radial side of the radius of curvature, whether provided with an angular offset or capable of being articulated to an offset orientation. First active electrode 730a may extend between 1-15 mm from the second portion planar surface. This distance may be selectable or preset. FIG. 7E illustrates another view of wand distal end 720 with the first active electrode portion 730a axially extended in the second configuration.

In alternative embodiments, these devices may operably couple and communicate with navigation means and robotics. Simple movement and easy controls for a variety of cut patterns could allow for different range of motion options. Hand control could include multiple axis of motion (side to side AND flexion/extension shown). In some other embodiments, the combination active electrode 730 may include a third active electrode (not shown), that may be similar to the first active electrode 730a in that it is axially positionable relative to second active electrode 730b. Inclusion of different material properties where the bending force (or shape memory materials) could be used for a variety of needs. Handles may be operated using a variety of actuation means such as a combination of triggers, thumb pushers (for the retraction or extension of the micro shaft), buttons, or knobs.

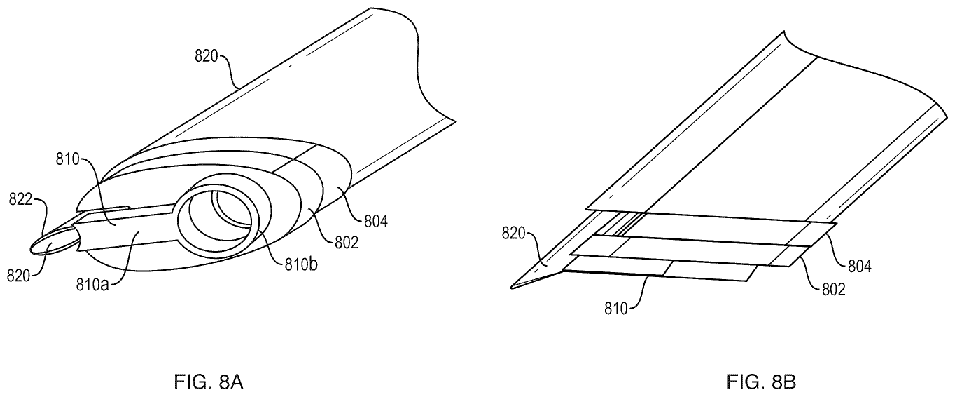

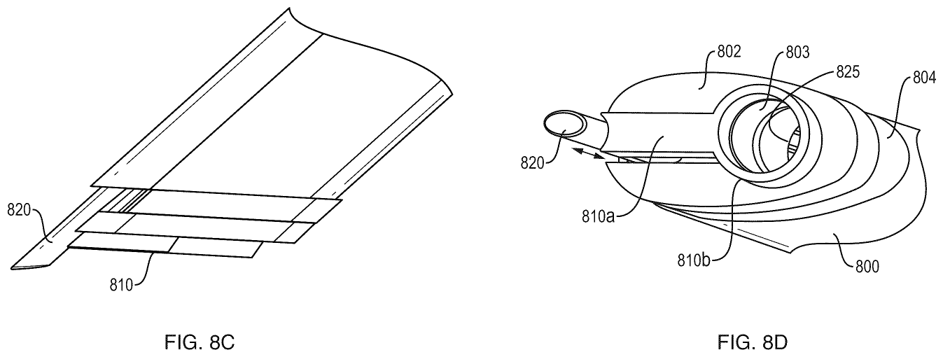

The specification now turns to FIGS. 8A-8D, showing various views of another example wand distal end embodiment 820, similar to the embodiment 720 described herein, except where noted. Embodiment 820 may be an articulating wand, or a fixed angular offset wand. Wand distal end 820 may include a multi-lumen shaft with a plurality of conduits therethrough, similar to wand distal end 720. Shaft cross section may be elongate or oval and include a return electrode collar 804 and insulative spacer 802. Spacer 802 is configured to electrically insulate the return electrode collar 804 from the active electrode (810 and 820). Spacer 802 may define a distal facing planar surface for supporting an second active electrode 810. Spacer 802 may have an oval shaped cross section, having a length greater than a width. Spacer 802 may have a suction opening 803 therethrough, in fluid communication with a fluid suction conduit that extends along the wand shaft.

Active electrode may include a first active electrode 820 and second active electrode 810. First active electrode 820 may be like other embodiments described herein in that it may be axially moveable relative to the second active electrode 810. First active electrode 820 may be a tubular member with an open end that is beveled 822. First active electrode 810 distal end edge may be continuous with a distal most surface of second active electrode 820 during debulking, as shown in FIG. 8B and axially advanced during fine dissection (FIG. 8C).

Second active electrode 810 distal-most surface may be planar and include a bridge portion 810a and an annular portion 810b. Second active electrode 810 may be symmetrically arranged about a plane bisecting the wand distal end, the bisecting plane parallel to the longest dimension of the distal end cross section. Bridge portion 810a may extend from the annular portion 810b to the first active electrode 820. Annular portion 810b may be radially offset from a longitudinal axis of wand. Annular portion 810b may be concentrically arranged with the spacer aspiration aperture 825 and may define an entrance aperture to the fluid aspiration conduit. Annular portion 810b may define a maximum cross sectional width portion of the entire active electrode in combination (810, 820), to maintain a large entrance aperture therethrough for efficient fluid and tissue removal.

Bridge portion 810a may electrically communicate with first active electrode 820. Bridge portion 810a may terminate with an opening or channel configured to slidingly receive at least a portion of the first active electrode 820 therethrough while maintaining electrical communication. First active electrode 820 may define a portion of outer peripheral surface of the entire active electrode, when combined with electrode 810. First active electrode 820 may define an outer edge portion of the wand distal end. First active electrode 820 may be connected to a spring, configured to bias a position of the first active electrode 820 in a retracted configuration. The user may then intentionally actuate an actuation means (such as a lever near the handle for example) to extend the first active electrode 820 beyond the second active electrode 810, and release of the actuation means may retract the first active electrode 820 via tension from the spring. This may prevent inadvertent tissue or wand damage.

The tubular member (first active electrode 820) may be a rigid member, and may have a limited axial length, limited by any bendable or articulating portion towards the wand distal end. For example, the tubular member may be 30 mm long. Articulation may therefore occur proximal from the entire tubular member. Compared with the first embodiment shown, this avoids overcoming friction associate with a shaft that slides along the entire shaft length. Tubular member (820) may be actuated by a small finger tab coupled to a proximal end edge of tubular member near distal end of the wand 800. In some embodiments tubular member may house a rod or wire (wire) that slides axially relative to the tubular member that may more finely dissect the target tissue. In some embodiments the tubular member may be solid in cross section. In some embodiments tubular member may define a channel for housing wiring in electrical communication with the active electrode (810, 820).

In some alternative embodiments bridge portion 810a may be recessed within spacer and may also be buried or covered to be entirely electrically insulated from tissue. In this embodiment, the exposed portions of the active electrode may include only the annular portion 810b, and first active electrode 820. When the first active electrode 820 is retracted, annular portion 810b may provide the sole debulking surface in this embodiment. When the first active electrode 220 is advanced, finer dissection may be limited to the first active electrode 820 when extended

Wand 820 may be plastically deformed to allow for articulation outside of the clinical space by the clinician for re-insertion and access to the location/tissue of interest or the articulation wire lines (2) shown above can be retracted during intraoperative use to allow for side-to-side motion (one wire at a time) or to allow for flexion (bending) (retracting both wires at the same time).

One skilled in the art will realize the disclosure may be embodied in other specific forms without departing from the spirit or essential characteristics thereof. The foregoing examples are therefore to be considered in all respects illustrative rather than limiting of the disclosure described herein. Scope of the disclosure is thus indicated by the appended claims, rather than by the foregoing description, and all changes that come within the meaning and range of equivalency of the claims are therefore intended to be embraced therein.

1. A bipolar electrosurgical wand, comprising:
a tubular end effector comprising an electrically insulative spacer, a return electrode and an active electrode at a distal end thereof, the insulative spacer supporting and electrically insulating the active electrode;
wherein the active electrode defines an annular portion and a tip projection extending distally from the annular portion, the annular portion coextensive with the insulative spacer and the tip projection extending distally from a distal-most surface of the insulative spacer, and wherein both the tip projection and annular portion share a continuous top planar surface;
wherein the annular portion includes a 360-degree bounded hole therethrough that defines an aspiration opening configured to remove at least one of tissue, tissue debris or fluid therethrough; wherein the aspiration opening defines a constant cross section and extends from the active electrode planar top surface through to a bottom surface of the active electrode at an incline angle to the planar top surface.

2. The bipolar electrosurgical wand of claim 1 wherein the tip projection has a maximum lateral width that is less than half a corresponding maximum lateral width of the annular portion.

3. The bipolar electrosurgical wand of claim 1 wherein the active electrode defines an outermost peripheral edge surface, that includes bilateral concave edge surfaces that are coextensive with each other at a transition from the annular portion to the tip projection and wherein the bilateral concave edge surfaces are coextensive with a distal-most end surface of the insulative spacer.

4. The bipolar electrosurgical wand of claim 1, wherein the aspiration opening is at least 15 degrees.

5. The bipolar electrosurgical wand of claim 1 wherein the incline angle is oriented such that an edge boundary of the aspiration opening at the bottom surface is axially offset, proximally from a corresponding edge boundary of the aspiration opening that is coincident with the top planar surface.

6. The bipolar electrosurgical wand of claim 5 wherein edge boundary at the bottom surface is configured to further digest aspirated tissue and tissue debris that flows through the aspiration opening.

7. The bipolar electrosurgical wand of claim 6 wherein the edge boundary at the bottom surface further comprises at least on notch configured to further digest aspirated tissue and tissue debris that flows through the aspiration opening.

8. The bipolar electrosurgical wand of claim 1 wherein the aspiration opening cross section has a first end, coincident with a bisecting plane of the active electrode that defines a first radius of curvature and an opposing end coincident with the bisecting plane that has a radius of curvature that is at least twice the first radius of curvature.

9. The bipolar electrosurgical wand of claim 8 wherein the first radius of curvature is configured to diminish a plasma-remote zone through the aspiration opening and wherein the second radius of curvature is configured to provide an expanded inner surface area for further digesting tissue and tissue debris that flows through the aspiration opening.

10. The bipolar electrosurgical wand of claim 1 wherein the return electrode has bilateral arms that extend around the distal-most end surface of the insulative spacer, defining distal facing surfaces of the return electrode coextensive with the active electrode tip projection, the bilateral arms configured to aid in plasma initiation at the tip projection.

11-17. (canceled)

18. A bipolar electrosurgical wand, comprising:
a tubular end effector having a handle at a proximal end and a return electrode, an insulative spacer and an active electrode at a distal end thereof, the insulative spacer supporting and electrically insulating the active electrode;
wherein the active electrode has an annular portion with a tip projection extending distally from the annular portion, the annular portion and the tip projection both sharing a continuous top planar surface;
wherein the annular portion defines a 360-degree bounded hole therethrough that defines an aspiration opening;
wherein the 360-degree bounded hole extends from the top planar surface to a bottom surface of the active electrode and defines a central axis that extends at an incline angle to the planar top surface, the incline angle configured to both further digest any tissue debris that flows through the aspiration opening and also deflect the tissue debris towards an aspiration conduit that extends proximally along the tubular end effector; and
wherein the return electrode has bilateral arms that extend around the distal-most end surface of the insulative spacer, defining distal facing surfaces of the return electrode coextensive with the active electrode tip projection, the bilateral arms configured to aid in plasma initiation at the tip projection.

19. The bipolar electrosurgical wand of claim 18 wherein the incline angle extends in a proximal direction from the planar top surface of the active electrode.

20. The bipolar electrosurgical wand of claim 18 wherein the 360-degree bounded hole defines a curved wedge cross section, with a proximal-most apex having a first radius of curvature and a distal-most curved end that has a radius of curvature that is at least double the first radius of curvature.

21. The bipolar electrosurgical wand of claim 18 wherein the tip projection has a maximum transverse width that is smaller than half of a maximum transverse width of the annular portion.

22. The bipolar electrosurgical wand of claim 18 wherein the tip projection defines a free end projection, that extends beyond the insulative spacer.

23. A method of electrosurgically treating a tissue along a patient airway comprising:
positioning an electrosurgical wand in a first orientation so that a planar top surface of an active electrode engages a first target tissue along the patient airway, the active electrode having an aspiration opening extending from the planar top surface to a bottom surface of the active electrode, the aspiration opening defining a central axis oriented at a non-perpendicular angle to the planar top surface so that a peripheral edge boundary of the aspiration opening at the bottom surface is axially offset from a corresponding peripheral edge boundary at the planar surface; and while the wand is in the first orientation;
applying electrical energy between the active electrode and a return electrode of the electrosurgical wand;
forming, responsive to the energy, a localized plasma proximate to the active electrode planar surface and debulking the first target tissue, by the localized plasma, to molecularly dissociate a portion of the first target tissue;
aspirating tissue and plasma by-products associated with the first target tissue through the aspiration opening; and
further molecularly dissociating the tissue and plasma by-products associated with the first target tissue via the localized plasma proximate to the peripheral edge boundary at the bottom surface the responsive to the energy.

24. The method of claim 23 further comprising:
placing the electrosurgical wand in a second orientation, such that a projecting tip of the active electrode is directly adjacent a second target tissue along the patient airway, the projecting tip defining a distal-most projection of the active electrode, extending parallel to and continuous with the planar top surface; and while the electrosurgical wand is in the second orientation;
applying electrical energy between the active electrode and the return electrode;
forming, responsive to the energy, a localized plasma proximate to the projecting tip and ablating, by the localized plasma, to finely dissect the second target tissue.

25. The method of claim 24 wherein applying electrical energy between the active electrode and the return electrode and forming, responsive to the energy, a localized plasma proximate to the projecting tip further comprises delivery electrically conductive fluid from a fluid delivery aperture proximally spaced from the active electrode along the wand distal end and around to a distal facing portion of the return electrode coextensive with the projecting tip, the distal facing portion and the projecting tip configured to reduce an electrical bridge burden on the electrically conductive fluid and thereby reduce a time to plasma initiation at the projecting tip.

26. The method of claim 24 wherein while applying electrical energy between the active electrode and a return electrode of the electrosurgical wand, protecting adjacent tissues from inadvertent thermal effects adjacent a back-side of the wand distal end formed of a ceramic thermal heat sink.

27. The method of claim 23 further comprising deflecting the tissue debris flowing through the aspiration opening proximally and towards an aspiration conduit disposed along the electrosurgical wand, the deflecting with a distal inner surface of the aspiration opening, the distal inner surface extending parallel to the central axis.

28-31. (canceled)
